(19) United States
(12) Patent Application Publication
(10) Pub. No.: US 2009/0254113 A1
(43) Pub. Date: Oct. 8, 2009

(54) Dilatation balloon with ridges and methods

(75) Inventors: Kevin Nolan, Loughrea (IE); Michael Cummins, Dysart Ballinasloe (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/062,717

(22) Filed: Apr. 4, 2008

(51) Int. Cl. A61M 29/00; B29C 41/46

(52) U.S. Cl. 606/194; 264/319

(57) ABSTRACT

The present invention provides a zero-fold dilatation balloon that includes a balloon body having a proximal end and a distal end and at least one ridge at the proximal end and at least one ridge at the distal end in an inflated state, wherein the balloon body between the ridges comprises a continuous polymer tube with an external surface having a hydrophilic coating thereon, and further wherein the balloon has a uniform profile along its entire length in a deflated state.

### BACKGROUND OF THE INVENTION

Surgical procedures employing balloons and medical devices incorporating those balloons (i.e., balloon catheters) are becoming more common and routine. These procedures, such as angioplasty procedures, are conducted when it becomes necessary to expand or open narrow or obstructed openings in blood vessels and other passageways in the body to increase the flow through the obstructed areas. For example, in an angioplasty procedure, a dilatation balloon catheter is used to enlarge or open an occluded blood vessel which is partially restricted or obstructed due to the existence of a hardened stenosis or buildup within the vessel. This procedure requires that a balloon catheter be inserted into the patient's body and positioned within the vessel so that the balloon, when inflated, will dilate the site of the obstruction or stenosis so that the obstruction or stenosis is minimized, thereby resulting in increased blood flow through the vessel.

Total or near-total occlusions in arteries can prevent all or nearly all of the blood flow through the affected arteries. It has been estimated that 5% to 15% of patients on whom percutaneous coronary angioplasty (PTCA) is attempted are found to have chronic total occlusions (CTO's) of at least one coronary artery. In patients who suffer from coronary CTO's, the successful performance of a PTCA is a technical challenge.

Balloons are typically tightly folded and wrapped upon themselves for delivery to the targeted lesion, and are unwrapped and expanded to a size that is considerably greater than the stored size by the introduction of an expansion fluid into the balloon, although zero-fold balloons are also known, such as that described in U.S. Pat. Pub. No. 2005/0118370. Such balloons have no folds or wraps.

Balloons can also be coated on the outside surface; however, this may lead to what is referred to in the art as “melon seeding.” This refers to slippage of the balloon wherein the balloon, which is too lubricious, shoots forward on inflation causing accidental slippage from the target (e.g., repair) site, which ultimately may lead to stent slippage from the target site as well.

It is therefore necessary to also find a way in which the balloon can be retained easily at the target site during expansion or contraction without slippage. This is more readily accomplished when the balloon has no lubricity. One method of overcoming this “melon seeding” effect is to make the balloons with both a lubricating portion and a non-lubricating portion. U.S. Pat. No. 5,503,631 (Onishi et al.) discloses a vasodilating catheter balloon whose body has a lubricating portion and a non-lubricating portion. The lubricious property of the balloon is created by grafting a lubricious coating onto a non-lubricious substrate. Only the tapered portions on opposite ends of the balloon were treated.

There is a continuing need in the industry for dilatation balloons that avoid the problems associated with the “melon seeding” effect.

### SUMMARY

The present invention provides zero-fold dilatation balloons, methods of making, and methods of using.

In one embodiment, the balloon includes a balloon body having a proximal end and a distal end and at least one ridge at the proximal end and at least one ridge at the distal end in an inflated state, wherein the balloon body between the ridges comprises a continuous polymer tube with an external surface having a hydrophilic coating thereon, and further wherein the balloon has a uniform profile (i.e., uniform outer diameter) along its entire length in a deflated state.

In certain embodiments, the balloon has one ridge at each of the proximal end and the distal end. In certain embodiments, the balloon body (in an inflated state) between the ridges is at least 6 millimeters (mm) in length. In certain embodiments, the balloon body between the ridges is no more than 30 mm in length. In certain embodiments, the ridges are at least 0.4 mm in diameter larger than the balloon body diameter between the ridges (in an inflated state). In certain embodiments, the ridges are no more than 0.5 mm in diameter larger than the balloon body diameter between the ridges. In certain embodiments, the ridges are at least 0.8 mm in length. In certain embodiments, the ridges are no greater than 1.2 mm in length. In certain embodiments, the ridges are 0.8 mm to 1.2 mm in length.

In certain embodiments, the balloon body between the ridges has a wall thickness that is the same as that of the ridges. In certain embodiments, the balloon includes one or more materials selected from the group consisting of polyethylene terephthalate homopolyester polymers and polybutylene terphthalate polymers. In certain embodiments, the balloon includes one or more thermoplastic polyurethane polymers. The polymer may or may not be crosslinked, but is preferably not crosslinked.

In another embodiment, the present invention provides a zero-fold dilatation balloon that includes: a balloon body having a proximal end and a distal end; and

one ridge at the proximal end and one ridge at the distal end in an inflated state, wherein the ridges are at least 0.4 mm in diameter larger than the balloon body diameter between the ridges; wherein the balloon body between the ridges is 6 mm to 30 mm in length and comprises a continuous polymer tube with an external surface having a hydrophilic coating thereon; and further wherein the balloon has a uniform profile along its entire length in a deflated state.

In another embodiment, the present invention provides a zero-fold dilatation balloon that includes: a balloon body having a proximal end and a distal end; and one ridge at the proximal end and one ridge at the distal end in an inflated state, wherein the ridges are at least 0.4 mm in diameter larger than the balloon body diameter between the ridges, and the ridges are 0.8 mm to 1.2 mm in length; wherein the balloon body between the ridges is 6 mm to 30 mm in length, has a wall thickness that is the same as that of the ridges, and comprises a continuous polymer tube with an external surface having a hydrophilic coating thereon; and further wherein the balloon has a uniform profile along its entire length in a deflated state.

The present invention also provides methods of making and using the dilatation balloons of the present invention.

In one embodiment, a method of reducing slippage of a dilatation balloon from a target site in a patient is provided. The method includes: providing a zero-fold dilatation balloon comprising: a balloon body having a proximal end and a distal end and at least one ridge at the proximal end and at least one ridge at the distal end in an inflated state; wherein the balloon body between the ridges comprises a continuous polymer tube with an external surface having a hydrophilic coating thereon; and further wherein the balloon has a uniform profile along its entire length in a deflated state; and inserting a balloon catheter comprising the balloon into the target site of the patient; and inflating the balloon and the ridges at the target site.

In another embodiment, the present invention provides a method of making a dilatation balloon. The method includes: providing a tubular parison comprising a polymeric material; providing a mold for forming a balloon with one or more ridges at each of the proximal and distal ends; expanding the tubular parison to form an expanded parison in the mold; providing a heat deflector in proximity to the expanded parison to shield a region between the ridges at the proximal and distal ends of the expanded parison; subjecting the expanded parison with the shielded region to a shrinkage process to form a zero-fold balloon having a uniform profile along its entire length in a deflated state, and comprising a balloon body having a continuous polymer tube with an external surface, at least one ridge at the proximal end, and at least one ridge at the distal end when in an inflated state; and applying a hydrophilic coating to the external surface of the continuous polymer tube between the regions at the proximal end and the distal end that form the ridges.

Preferably, expanding the tubular parison to form an expanded parison comprises axially stretching and radially expanding the tubular parison at a temperature above the Tg of the polymeric material and at an elevated inflation pressure; and subjecting the expanded parison with the shielded region to a shrinkage process comprises: heating the expanded parison to a temperature above the temperature at which the balloon was axially stretched and radially expanded, but below the melting temperature of the polymeric material of the tubular parison; and reducing the inflation pressure to 0 psi; wherein the shrinkage process is carried out for a time sufficient to form a zero-fold balloon having a uniform profile along its entire length in a deflated state.

Herein, the terms “distal” and “proximal” are used with respect to a position or direction relative to the treating clinician. “Distal” and “distally” are a position distant from or in a direction away from the clinician. “Proximal” or “proximally” are a position near or in a direction toward the clinician.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably.

As used herein, the term “or” is generally employed in its sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is a representation of a mold for making a balloon of the present invention and is shown with exemplary dimensions.

FIG. 2 shows the balloon catheter in an inflated state with balloon (108) showing ridges (207) at either end.

FIG. 3 shows the distal section (100) of the balloon catheter with the balloon (108) in an inflated state showing ridges (207).

FIG. 4 shows the balloon catheter in a deflated state.

FIG. 5 shows the distal section (500) of the balloon catheter with the balloon in a deflated state.

FIG. 6 is a cross-sectional view of the central portion of the deflated balloon of FIG. 4.

### DETAILED DESCRIPTION OF ILLUSTRATIVE EMBODIMENTS

The present invention provides a zero-fold dilatation balloon, methods of making, and methods of using.

The balloon includes a balloon body having a proximal end and a distal end and at least one ridge at the proximal end and at least one ridge at the distal end in an inflated state. The balloon body between the ridges includes a continuous polymer tube having a hydrophilic coating. Furthermore, the balloon has a uniform profile (i.e., uniform outer diameter) along its entire length in a deflated state. Such ridges anchor the balloon to reduce and/or prevent “melon seeding,” i.e., slippage of the balloon from the target (e.g., repair) site.

Balloons of the present invention include a balloon body having a proximal end and a distal end and at least one ridge at the proximal end and at least one ridge at the distal end in an inflated state. Herein, “at the proximal end” and “at the distal end” means that the ridges form the ends of the balloon, or one or more ridges is no further than 5 mm from each end of the balloon. Although there can be more than one ridge at each end of the balloon, only one at each end is required to function as an anchor. Dimensions provided herein apply to the balloon when it is in a fully inflated state, unless otherwise specified.

Balloons of the present invention have a balloon body between ridges that includes a continuous polymer tube. In certain embodiments, the length of the balloon body (in an inflated state) between the ridges (i.e., the continuous polymer tube) is at least 6 mm in length. In certain embodiments, the length of the balloon body between the ridges is no more than 30 mm in length.

In certain embodiments, the diameter of the balloon body (in an inflated state) between the ridges (i.e., the continuous polymer tube) is at least 1.0 mm. In certain embodiments, the diameter of the balloon body between the ridges is no more than 1.5 mm. Typically, the diameter of the balloon body between the ridges is on average 1.25 mm.

The diameter of the balloon body at each ridge (in an inflated state) may be the same or different. Preferably, the diameter of the balloon body at the ridges is at least 0.4 mm in diameter larger, and more preferably no more than 0.5 mm in diameter larger, than the balloon body diameter between the ridges. In certain embodiments, the diameter of the balloon body at the ridges is at least 1.4 mm. In certain embodiments, the diameter of the balloon body at the ridges is no more than 2.0 mm. Typically, the diameter of the balloon body at the ridges is on average 1.65 to 1.75 mm.

The length of the balloon body at each ridge (in an inflated state) may be the same or different. In certain embodiments, the length of the balloon body at the ridges is at least 0.8 mm. In certain embodiments, the diameter of the balloon body at the ridges is no more than 1.2 mm.

Balloons of the present invention have a balloon body between the ridges that includes a continuous polymer tube with a wall thickness that is typically the same as that of the ridges in a deflated state. In certain embodiments, the wall thickness of the balloon body between the ridges is at least 0.012 mm. In certain embodiments, the wall thickness of the balloon body between the ridges is no more than 0.025 mm. In certain embodiments, the wall thickness of the balloon body at the ridges is at least 0.012 mm. In certain embodiments, the wall thickness of the balloon body at the ridges is no more than 0.025 mm. When inflated to nominal pressure, the ridges appear (or reappear) and, typically, have lower wall thickness than the body between them. Also, the balloon wall thickness is lower than the thickness of an associated catheter shaft.

Balloons of the present invention are zero-fold. The phrase zero-fold is used herein to refer to balloons that have no folds or wraps.

Balloons of the present invention may be compliant, noncompliant, or semi-compliant. This classification is based upon the operating characteristics of the individual balloon, which in turn depend upon the process used in forming the balloon, as well as the material used in the balloon forming process. All types of balloons provide advantageous qualities. A balloon which is classified as “noncompliant” is characterized by the balloon's inability to grow or expand appreciably beyond its rated or nominal diameter. Noncompliant balloons are referred to as having minimal distensibility. In balloons currently known in the art (e.g., polyethylene terephthalate), this minimal distensibility results from the strength and rigidity of the molecular chains which make up the base polymer, as well as the orientation and structure of those chains resulting from the balloon formation process.

A balloon which is referred to as being “compliant” is characterized by the balloon's ability to grow or expand beyond its nominal or rated diameter. In balloons currently known in the art (e.g., polyethylene, polyvinylchloride), the balloon's compliant nature or distensibility results from the chemical structure of the polymeric material used in the formation of the balloon, as well as the balloon forming process. Compliant balloons upon subsequent inflations, will achieve diameters which are greater than the diameters which were originally obtained at any given pressure during the course of the balloon's initial inflation.

A balloon which is referred to as being “semi-compliant” is characterized by low compliance with moderate stretching upon the application of tensile force. Typically, a semi-compliant balloon has a compliance of less than 0.045 millimeters/atmosphere (mm/atm), whereas a compliant balloon has a compliance of greater than 0.045 mm/atm, and a noncompliant balloon has a compliance of not greater than 0.025 mm/atm. Examples of such semi-compliant balloon materials include Nylon 12 and Pebax 7033.

Dimensions provided herein are the dimensions of the balloon when it is in a fully inflated state and at its nominal or rated diameter (i.e., upon initial inflation for a compliant balloon), unless otherwise specified.

Preferred balloons of the present invention have high elasticity and high elastic recovery. Preferably, the balloon returns to approximately the same profile it had before the initial inflation.

The term “elastic,” as it is used in connection with this invention, refers only to the ability of a material to follow the same stress-strain curve upon the multiple applications of stress. Elasticity, however, is not necessarily a function of how distensible a material is. It is possible to have an elastic, non-distensible material or a nonelastic, distensible material.

Before initial inflation and when deflated, balloons of the present invention preferably have a much lower profile than wrapped conventional balloons, and can have essentially the same dimensions as the tubular pre-form. When inflated, balloons of the present invention transition from a low profile tube to a balloon having ridges at the proximal and distal ends. They preferably revert to the initial tubular form when deflated, even after multiple inflations and after multiple lesions have been dilated. Balloons of the present invention have elasticity at nominal strains of at least 30%. Alternatively, balloons of the present invention have elastic recovery from nominal strains equal to, or greater than, 30%, 40%, 50%, 60%, 70%, 80%, 90%, or 100%, where nominal strain is [(balloon o.d. at nominal pressure-preform o.d.)/preform o.d.]×100, where “o.d.” is the outer diameter. Preferred balloons of the present invention may, therefore, be used to dilate multiple lesions without compromising primary performance.

Materials used in balloons of the present invention are primarily thermoplastics or thermoplastic elastomers. They may be block co-polymers, graft co-polymers, a blend of elastomers and thermoplastics, and the like. Such polymers may be crosslinked or not, but preferably are not crosslinked. Various combinations of polymers may be used in making balloons of the present invention. Exemplary materials include polyesters and copolymers thereof, polyamides and copolymers thereof, polyethylenes and copolymers thereof, and polyurethanes and copolymers thereof. Typically, and preferably, such polymers are block copolymers. Examples of mixtures of polymers include mixtures of nylon and polyamide block copolymers and polyethylene terephthalate and polyester block copolymers.

For example, the polymers may include polyethylene terephthalate polymers and polybutylene terphthalate polymers. Other useful materials include polyesterether and polyetheresteramide copolymers such as those described in U.S. Pat. No. 5,290,306 (Trotta et al.), polyether-polyamide copolymers such as those described in U.S. Pat. No. 6,171,278 (Wang et al.), polyurethane block copolymers such as those described in U.S. Pat. Nos. 6,210,364 B1, 6,283,939 B1, and 5,500,180 (all to Anderson et al.). Suitable polymers also include materials such as the multiblock copolymers of the zero-fold balloon described in U.S. Pat. Pub. No. 2005/0118370.

A particularly preferred block copolymer which can be used in accordance with the process of this invention is polyurethane block copolymer. This preferred polymer may be made, for example, by a reaction between a) an organic diisocyanate; b) a polyol; and c) at least one chain extender. Preferred polyurethanes which can be used in this invention may be varied by using different isocyanates and polyols which will result in different ratios of hard to soft segments as well as different chemical interactions within the individual regions of the polymer. They may include polyurethanes available under the trade designation PELLETHANE 2363-75D and polyether block amide copolymers available under the trade designation PEBAX 7033. Preferably, the polyurethane is manufactured by the Dow Chemical Company and marketed under the trade name PELLETHANE 2363-75D. This raw material has a Shore Hardness of about 74 D, a specific gravity of about 1.21, a tensile modulus of about 165,000 pounds per square inch (psi), a flexural modulus of about 190,000 psi, an ultimate tensile strength of about 6,980 psi, and an ultimate elongation of about 250%.

Balloons of the present invention have a balloon body between ridges that includes a continuous polymer tube having a hydrophilic coating thereon to decrease the friction between sliding surfaces. Such hydrophilic coating is typically applied to the continuous polymer tube between the regions that form the ridges by masking these regions, coating the hydrophilic material on the continuous polymer tube as is done conventionally in the art, and removing the masking material to expose the uncoated regions. This can be done when the balloon is in the inflated state or in the uninflated state. If necessary, such coating material can be cured using radiation, such as ultraviolet light.

Exemplary materials for the hydrophilic coating include PhotoLink™ lubricity coating made by SurModics, Inc.

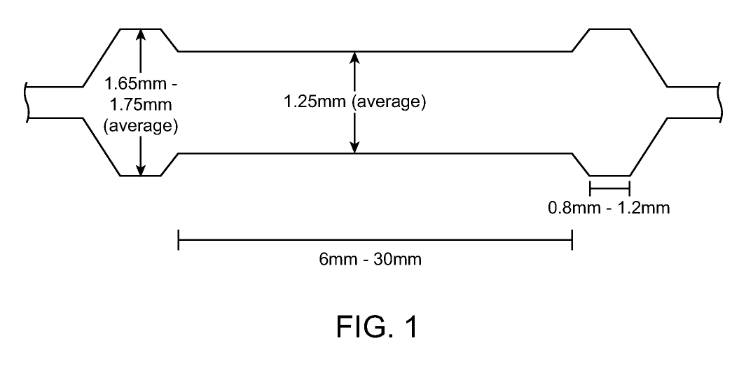

In accordance with this invention, the balloons are formed from a thin wall parison of a polymeric material, preferably made of a polyurethane block copolymer, using a mold which can be provided with a heating element. An exemplary mold that is capable of forming one ridge at each of the proximal and distal ends with exemplary dimensions is shown in FIG. 1.

In a preferred embodiment, the mold receives a tubular parison made of a polymeric material. The ends of the parison extend outwardly from the mold and one of the ends is sealed while the other end is affixed to a source of inflation fluid, typically nitrogen gas, under pressure. Clamps or “grippers” are attached to both ends of the parison so that the parison can be drawn apart axially in order to axially stretch the parison while at the same time said parison is capable of being expanded radially or “blown” with the inflation fluid. The radial expansion and axial stretch step or steps may be conducted simultaneously, or depending upon the polymeric material of which the parison is made, following whatever sequence is required to form a balloon. Failure to axially stretch the parison during the balloon forming process will result in a balloon that will have an uneven wall thickness and will exhibit a wall tensile strength lower than the tensile strength obtained when the parison is both radially expanded and axially stretched.

The polymeric parisons used in this invention are preferably drawn axially and expanded radially simultaneously within the mold. To improve the overall properties of the balloons formed, it is desirable that the parison is axially stretched and blown at temperatures above the glass transition temperature (Tg) of the polymeric material used. This expansion usually takes place at a temperature of 80° C. to 150° C., depending upon the polymeric material used in the process.

In accordance with this invention, based upon the polymeric material used, the parison is dimensioned with respect to the intended final configuration of the balloon. It is particularly important that the parison have relatively thin walls. The wall thickness is considered relative to the inside diameter of the parison which has wall thickness-to-inside diameter ratios of less than 0.6, and preferably between 0.57 and 0.09 or even lower. The use of a parison with such thin walls enables the parison to be stretched radially to a greater and more uniform degree because there is less stress gradient through the wall from the surface of the inside diameter to the surface of the outside diameter. By utilizing a parison which has thin walls, there is less difference in the degree to which the inner and outer surfaces of the tubular parison are stretched.

Preferably, the parison is drawn from a starting length L1 to a drawn length L2, which preferably is between about 1.10 to about 6 times the initial length L1. The tubular parison, which has an initial internal diameter ID1 and an outer diameter OD1, is expanded by the inflation fluid emitted under pressure to the parison to an internal diameter ID2, which is preferably 6 to 8 times the initial internal diameter ID1, and an outer diameter OD2, which is about equal to or preferably greater than about 3 times the initial outer diameter OD1. The parison is preferably subjected to between 1 and 5 cycles during which the parison is axially stretched and radially expanded with an elevated inflation pressure (i.e., a pressure sufficient to inflate the balloon), preferably an elevated pressure of at least 100 psi, and more preferably up to 500 psi. Nitrogen gas is the preferable inflation fluid for the radial expansion step.

Following the initial expansion step, the expanded parison is subjected to a “Heat Set” step, preferably while maintaining the elevated inflation pressure of at least 100 psi and more preferably up to 500 psi. The temperature chosen for the “Heat Set” step is one that induces crystallization and “freezes” or “locks” the orientation of the polymer chains which resulted from axially stretching and radially expanding the parison. The temperatures which can be used in this heat set step are therefore dependent upon the particular polymeric material used to form the parison and the ultimate properties desired in the balloon product (e.g., distensibility, strength, and compliancy). The temperatures chosen for this “Heat Set” step will more usually be above the temperature used during the initial expansion step but will be below the melting temperature of the melt temperature of the polymeric material from which the parison is formed. The heat set step ensures that the expanded parison and the resulting balloon will have temperature and dimensional stability.

After the balloon has been formed in the mold and following the “Heat Set” step, and while still axially restrained, the expanded parison is subjected to a shrinkage process in which the body of the balloon is exposed to less heat than the ridges at the proximal and distal ends, thereby shrinking the ends relative to the balloon working length. During this process a heat deflector (e.g., a material with poor heat conducting characteristics such as PEEK (polyether ether ketone)) is used in proximity to a region between the proximal and distal ends of the expanded parison (i.e., in proximity to the region of the balloon body between the ridges formed in the mold of FIG. 1, for example) to shield the balloon body (between the proximal and distal ends) from heat applied during the shrinkage process. This results in the body of the balloon that is shielded to be exposed to less heat than the regions at the proximal and distal ends during the shrinkage process. The unshielded regions thereby form the ridges upon inflation. The heat in the shrinkage process is applied for a time sufficient for the expanded parison to shrink to a uniform profile along its length. During this shrinkage process the inflation pressure is reduced to 0 psi for a time (e.g., 30 seconds) sufficient to achieve the required shrinkage to produce a zero-fold balloon. Thus, the method of the present invention preferentially shrinks the ends of the balloon by shielding the central portion of the balloon working length from the heat source during shrinkage.

After the shrinkage step is completed, and while the parison is still axially restrained, the mold is cooled to room temperature or at least to less than 37° C. The finished balloon will typically obtain its rated or nominal diameter when inflated to a pressure of 5 bars to 8 bars depending upon the polymeric material used to form the balloon. A preferred balloon has a nominal diameter at 10 atmospheres (atm).

If the parison is formed from the polyurethane marketed by The Dow Chemical Company under the trade name PELLETHANE 2363-75D and axially stretched and radially expanded at a temperature of 90-100° C., the heat set step would preferably be conducted at about 105-120° C. If this step is conducted at temperatures much above 120° C., the tensile strength of the resulting polyurethane balloon would decrease significantly. Moreover, if the heat set step is conducted at temperatures significantly higher than 120° C., the distensibility of the resulting polyurethane balloon would also be adversely affected. However, if the heat set is conducted at temperatures below 100° C., the polyurethane balloons formed would be dimensionally unstable resulting in balloons with uneven wall thicknesses. Additionally, the lower heat set temperature would result in balloons exhibiting physical properties that would more likely be adversely affected during sterilization. Typical sterilization processes used for balloon catheters can be used to sterilize the balloons of the present invention.

The balloon thus formed may be removed from the mold, and affixed to a catheter. Following balloon formation, and prior to mounting on the catheter, one taper/cone region of the balloon is trimmed completely off the balloon (distal balloon region) while the other taper/cone region remains to form one of the bond regions. The other bond region of the balloon is part of the balloon body.

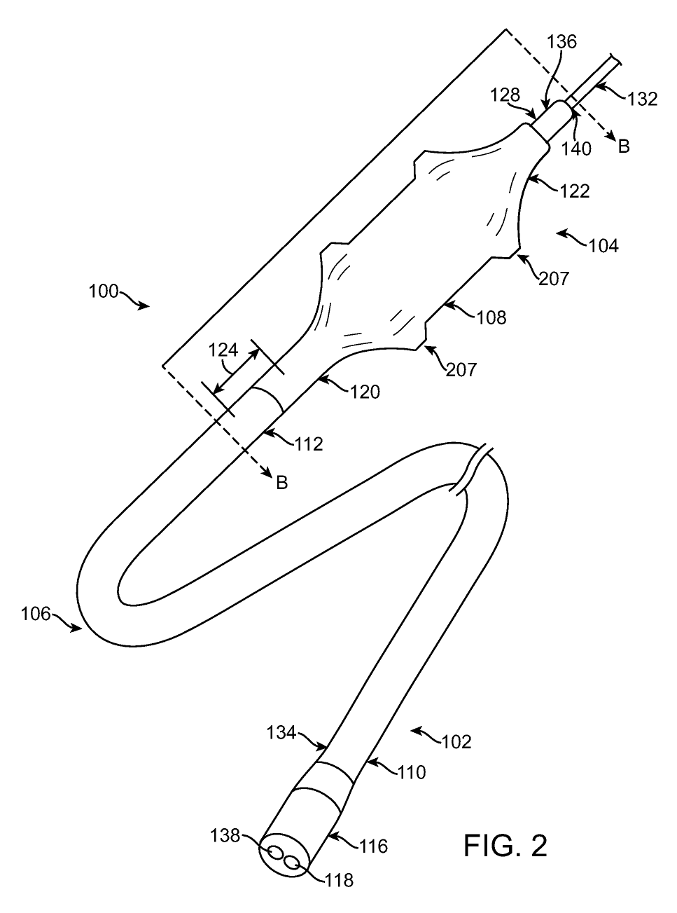

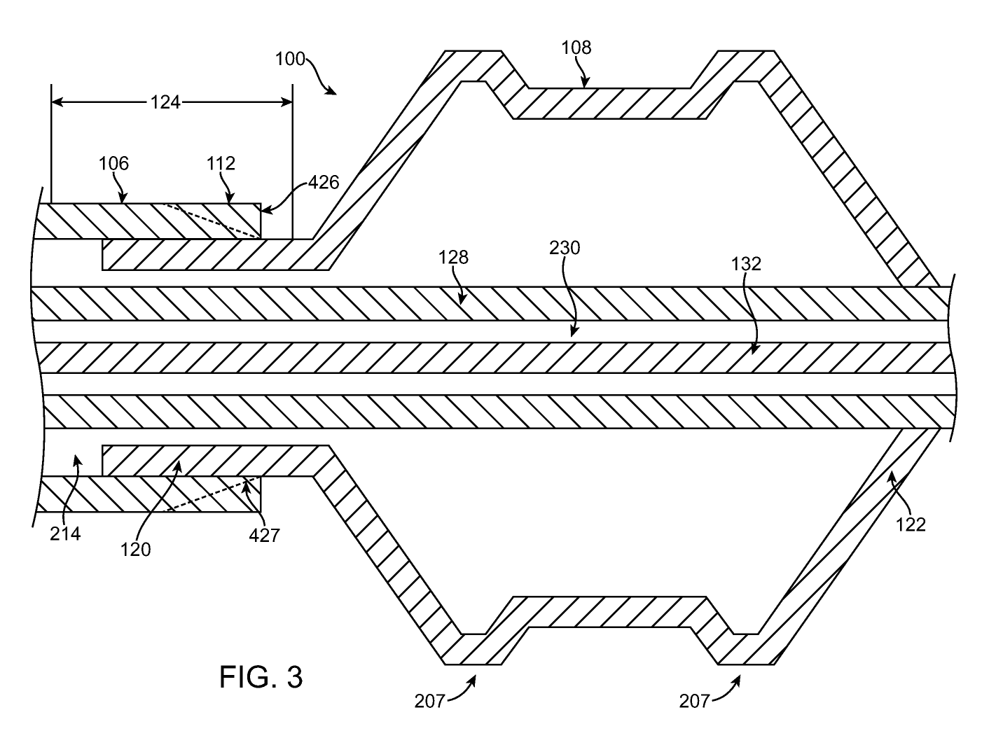

Referring now to FIGS. 2-3, an embodiment of a balloon catheter 100 according to the present invention is shown in an inflated state showing the ridges 207. Balloon catheter 100 includes a proximal portion 102, a distal portion 104, and an inflatable balloon 108 located at distal portion 104. Catheter 100 may be used for angioplasty procedures, stent delivery, and/or localized drug delivery.

Catheter 100 includes an outer catheter shaft 106 which includes at least one continuous lumen 214 extending from at or near its proximal end 110 to at or near its distal end 112 in order to provide for balloon inflation. Balloon 108 is located at or near distal end 112 of shaft 106, and a hub 116 is located at or near proximal end 110 of shaft 106. Hub 116 includes a balloon inflation port 118 to allow fluid communication between inflation lumen 214 and balloon 108 so that the balloon 108 may be inflated. Hub 116 will serve in a conventional manner to provide a luer or other fitting in order to connect the catheter 100 to a source of balloon inflation, such as conventional angioplasty activation device.

Balloon 108 includes a proximal end 120 and a distal neck end 122 and ridges 207. At joint transition area 124, proximal end 120 of balloon 108 is placed inside and joined to the distal end 112 of outer catheter shaft 106, as shown in FIG. 3. Balloon 108 may be joined to outer catheter shaft 106 in any conventional manner, such as laser welding, adhesives, heat fusing, ultrasonic welding, or any other mechanical method. The profile of balloon catheter 100 is reduced by placing the proximal end 120 of balloon 108 inside outer catheter shaft 106 because such a configuration allows for a smaller outer diameter at joint transition area 124.

FIG. 3 is an enlarged sectional view at the location along line B-B of FIG. 2, and illustrates joint transition area 124 of catheter 100. As previously mentioned, typically an angioplasty balloon is welded or otherwise mechanically attached to the outer catheter shaft by placing the proximal balloon neck on the outside of the catheter shaft. By placing the proximal balloon neck on the outside of the catheter shaft, the catheter presumably possesses a smoother profile for tracking the balloon to the treatment site since the “edge” created by the balloon to shaft joint is not pushed against the vessel wall while the balloon is being tracked through the patient's tortuous anatomy. However, it is found that the edge 426 created by proximal end 120 of balloon 108 being placed inside the outer catheter shaft 106 will not hinder the crossability and trackability of catheter 100 while balloon 108 is being tracked through the patient's tortuous anatomy. Rather, having the proximal end 120 of balloon 108 placed inside the outer catheter shaft allows for a smaller outer diameter at joint transition area 124 and thus provides a reduced catheter profile with improved crossability, trackability and stiffness.

In addition, edge 426 may be modified in order to create a tapered edge 427. Tapered edge 427 is illustrated as a dotted line in FIG. 3. Tapered edge 427 creates a smoother joint transition area 124 to ensure that the distal edge of the catheter shaft is not pushed against the vessel wall while being tracked through the patient's tortuous anatomy. Edge 426 may also be rounded or otherwise modified such as by a necking or thinning operation to create a smoother joint transition area 124.

In this exemplary embodiment of FIGS. 2-3, the inflated balloon body (108) between the ridges (207) is from 6 mm to 30 mm long; the diameter of the balloon body between the ridges is from 1 mm to 1.5 mm and is on average 1.25 mm; the diameter of the ridges (207) is from 0.4 mm to 0.5 mm greater than the diameter of the balloon body between the ridges; the diameter of the balloon at the ridges is not greater than 2.0 mm, and typically is from 1.65 mm to 1.75 mm; and the length of the constant diameter portion of the ridges is from 0.8 mm to 1.2 mm in length.

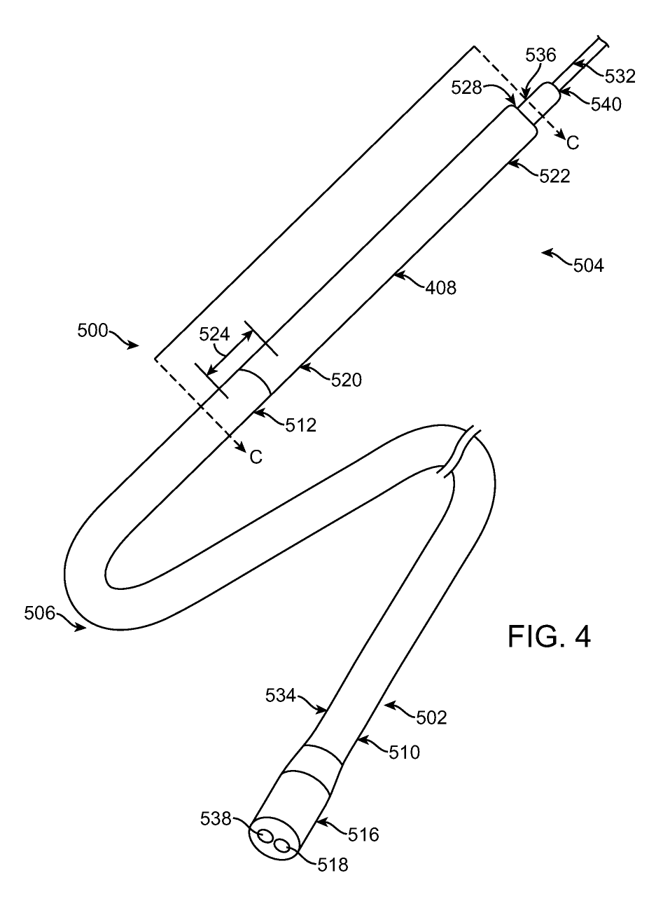

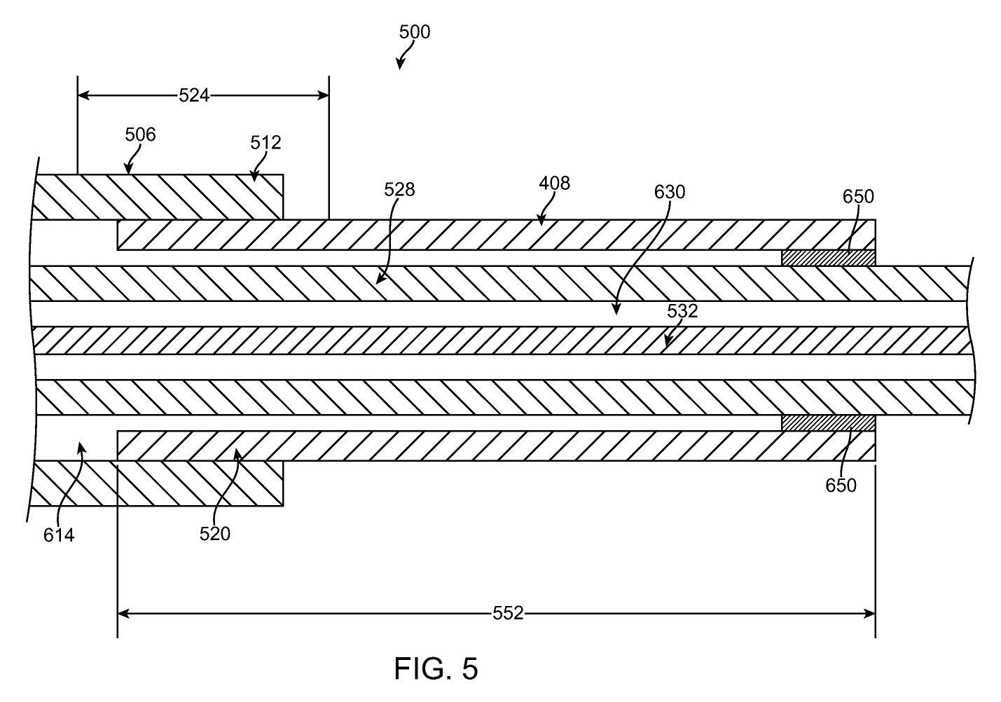

Now referring to FIGS. 4-6, another aspect of the present invention relates to a catheter 500 including a balloon 408 bonded to an outer catheter shaft 506, wherein the balloon is shown in a deflated state. FIG. 4 illustrates balloon catheter 500 having a proximal portion 502 and a distal portion 504 with inflatable balloon 408 located at distal portion 504. As best shown in FIG. 5, balloon 408 has a length 552. In addition to forming the basis for balloon angioplasty procedures, catheter 500 may form the basis of a stent delivery system and/or a drug delivery system.

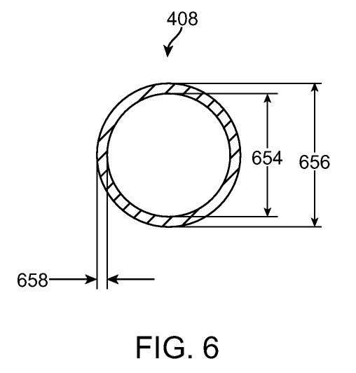

FIG. 6 is a cross-sectional view of the balloon of FIG. 4 in a deflated state, and illustrates that balloon 408 has a wall thickness 658, an inner diameter 654, and an outer diameter 656. In an unexpanded (i.e., uninflated) configuration, wall thickness 658, inner diameter 654, and outer diameter 656 are uniform along the full length 552 of balloon 408. A balloon with such uniform dimensions provides for a more flexible balloon by eliminating the thicker neck and taper portions of the balloon. In addition, a balloon with such uniform dimensions is not folded prior to inflation, but is instead expanded to the working diameter from a generally cylindrical or tubular shape. This no-fold aspect of balloon 408 also reduces the profile of catheter 500, thus resulting in improved crossability and trackability.

Preferably, FIG. 6 illustrates an exemplary embodiment in which the balloon (408), in a deflated state, has a wall thickness (658) that ranges from 0.012 mm to 0.025 mm, an inner diameter (654) that ranges from 0.5 mm to 0.8 mm??, and an outer diameter (656) that ranges from 0.6 mm to 0.9 mm. In an unexpanded (i.e., deflated) configuration, wall thickness (658), inner diameter (654), and outer diameter (656) are uniform along the full length (552) of balloon (408).

Catheter 500 includes outer catheter shaft 506 which includes at least one continuous lumen 614 extending from at or near its proximal end 510 to at or near its distal end 512 in order to provide for balloon inflation. Balloon 408 is located at or near distal end 512 of shaft 506, and a hub 516 is located at or near proximal end 510 of shaft 506. Hub 516 includes a balloon inflation port 518 to allow fluid communication between inflation lumen 614 and balloon 408 so that the balloon 408 may be inflated. Hub 516 will serve in a conventional manner to provide a luer or other fitting in order to connect the catheter 500 to a source of balloon inflation, such as conventional angioplasty activation device.

FIG. 5 is an enlarged sectional view at the location along line C-C of FIG. 4, and illustrates joint transition area 524 of catheter 500. Balloon 408 includes a proximal end 520 and a distal end 522. At joint transition area 524, proximal end 520 of balloon 408 is placed inside and joined to the distal end 512 of outer catheter shaft 506. Balloon 408 may be joined to outer catheter shaft 506 in any conventional manner, such as laser welding, adhesives, heat fusing, ultrasonic welding, or any other mechanical method. The profile of balloon catheter 500 is reduced by placing the proximal end 520 of balloon 408 inside outer catheter shaft 506 because such a configuration allows for a smaller outer diameter at joint transition area 524. Transition area 524 in FIG. 5 may also be rounded or otherwise modified such as by a necking or thinning operation to create a smoother transition joint.

Catheter 500 includes an inner or guidewire shaft 528 disposed coaxially within outer catheter shaft 506. Inner shaft 528 includes at least one continuous lumen 630 extending from at or near its proximal end 534 to at or near its distal end 536 in order to provide a guidewire lumen 532. As illustrated in FIG. 4, inner shaft 528 may extend the entire length of catheter 500, with a proximal guidewire port 538 provided in hub 516 and a distal guidewire port 540 provided at the distal portion of catheter 500. The distal end 522 of balloon 408 is joined to the inner shaft 528 at joint 650 (FIG. 5). Balloon 508 may be joined to inner shaft 528 in any conventional manner, such as laser welding, adhesives, heat fusing, ultrasonic welding, or any other mechanical method.

The embodiments illustrated in FIGS. 2-6 include inner shaft (128 or 528) disposed within outer catheter shaft (106 or 506), with inner shaft (128 or 528) extending the entire length of catheter (100 or 500). Such a configuration is typically referred to as an over-the-wire (OTW) catheter. An OTW catheter's guidewire shaft runs the entire length of the catheter and is attached to, or enveloped within, an inflation shaft. Thus, the entire length of an OTW catheter is tracked over a guidewire during a PTCA procedure.

One skilled in the art can appreciate how the balloon to catheter joint of the present invention, described in detail above, may also be incorporated in a rapid exchange (RX) catheter. A RX catheter has a guidewire shaft that extends within only the distalmost portion of the catheter. Thus, during a PTCA procedure only the distalmost portion of a RX catheter is tracked over a guidewire.

Outer catheter shaft (106 or 506) may be formed of any appropriate polymeric material. In addition, inner shaft (128 or 528) may be made of any appropriate polymeric material. Non-exhaustive examples of material for outer catheter shaft (106 or 506) and inner shaft (128 or 528) include polyethylene, PEBAX, nylon or combinations of any of these, either blended or co-extruded. Preferred materials for shafts (106 or 506 and 128 or 528) are polyethylene, nylon, PEBAX, or co-extrusions of any of these materials.

Optionally, shafts (106 or 506 and 128 or 528) or some portion thereof may be formed as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. For example, at least a proximal portion of outer catheter shaft 106 may in some instances be formed from a reinforced polymeric tube. As a further alternative, at least a proximal portion of outer catheter shaft (106 or 506) may in some instances be formed from a metal, highly elastic, or super elastic hypotube material.

Referring to FIG. 4, balloon 408 with such uniform dimensions as described above is not folded prior to inflation, but is instead expanded to the working diameter from a generally cylindrical or tubular shape. This no-fold aspect of balloon 408 reduces the profile of catheter 500 during insertion, thus resulting in improved crossability and trackability. Once balloon 408 is inflated, it assumes the shape of balloon 108 shown in FIG. 2 in order to enlarge the lumen of the affected coronary artery. Upon deflation, elastic shrinkage of the working outer diameter of the balloon occurs such that balloon 408 reduces in OD and catheter 500 may be retracted from the patient.

In any of the embodiments shown herein, inner shaft (e.g., 528 in FIG. 4) and outer catheter shaft (e.g., 506 in FIG. 4) may be arranged in various dual lumen configurations. For example, inner shaft and outer catheter shaft may be arranged in a coaxial dual lumen configuration. In the coaxial dual lumen configuration, an inflation lumen is created by a space between the outer surface of inner shaft and the inner surface of outer catheter shaft. This inflation lumen is in fluid communication with an interior of balloon such that balloon may be inflated. Other embodiments of balloon catheter may have guidewire lumen and inflation lumen in other dual lumen arrangements, such as a circular guidewire lumen above a D-shaped inflation lumen or a circular guidewire lumen set above a crescent-shaped inflation lumen.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A zero-fold dilatation balloon comprising:
a balloon body having a proximal end and a distal end and at least one ridge at the proximal end and at least one ridge at the distal end in an inflated state;
wherein the balloon body between the ridges comprises a continuous polymer tube with an external surface having a hydrophilic coating thereon; and
further wherein the balloon has a uniform profile along its entire length in a deflated state.

2. The balloon of claim 1 having one ridge at each of the proximal end and the distal end.

3. The balloon of claim 1 wherein the balloon body between the ridges is at least 6 mm in length.

4. The balloon of claim 1 wherein the balloon body between the ridges is no more than 30 mm in length.

5. The balloon of claim 1 wherein the ridges are at least 0.4 mm in diameter larger than the balloon body diameter between the ridges.

6. The balloon of claim 1 wherein the ridges are no more than 0.5 mm in diameter larger than the balloon body diameter between the ridges.

7. The balloon of claim 1 wherein the ridges are at least 0.8 mm in length.

8. The balloon of claim 1 wherein the ridges are no greater than 1.2 mm in length.

9. The balloon of claim 1 wherein the balloon body between the ridges has a wall thickness that is the same as that of the ridges.

10. The balloon of claim 1 comprising one or more materials selected from the group consisting of polyethylene terephthalate homopolyester polymers and polybutylene terphthalate polymers.

11. The balloon of claim 1 comprising one or more thermoplastic polyurethane polymers.

12. A zero-fold dilatation balloon comprising:
a balloon body having a proximal end and a distal end; and
one ridge at the proximal end and one ridge at the distal end in an inflated state, wherein the ridges are at least 0.4 mm in diameter larger than the balloon body diameter between the ridges;
wherein the balloon body between the ridges is 6 mm to 30 mm in length and comprises a continuous polymer tube with an external surface having a hydrophilic coating thereon; and
further wherein the balloon has a uniform profile along its entire length in a deflated state.

13. The balloon of claim 12 wherein the ridges are 0.8 mm to 1.2 mm in length.

14. The balloon of claim 12 wherein the balloon body between the ridges has a wall thickness that is the same as that of the ridges.

15. The balloon of claim 12 comprising one or more thermoplastic polyurethane polymers.

16. A zero-fold dilatation balloon comprising:
a balloon body having a proximal end and a distal end; and
one ridge at the proximal end and one ridge at the distal end in an inflated state, wherein the ridges are at least 0.4 mm in diameter larger than the balloon body diameter between the ridges, and the ridges are 0.8 mm to 1.2 mm in length;
wherein the balloon body between the ridges is 6 mm to 30 mm in length, has a wall thickness that is the same as that of the ridges, and comprises a continuous polymer tube with an external surface having a hydrophilic coating thereon; and
further wherein the balloon has a uniform profile along its entire length in a deflated state.

17. The balloon of claim 16 comprising one or more materials selected from the group consisting of polyethylene terephthalate homopolyester polymers and polybutylene terphthalate polymers.

18. The balloon of claim 16 comprising one or more thermoplastic polyurethane polymers.

19. A method of reducing slippage of a dilatation balloon from a target site in a patient, the method comprising:
providing a zero-fold dilatation balloon comprising:
a balloon body having a proximal end and a distal end and at least one ridge at the proximal end and at least one ridge at the distal end in an inflated state;
wherein the balloon body between the ridges comprises a continuous polymer tube with an external surface having a hydrophilic coating thereon; and
further wherein the balloon has a uniform profile along its entire length in a deflated state; and
inserting a balloon catheter comprising the balloon into the target site of the patient; and
inflating the balloon and the ridges at the target site.

20. The method of claim 19 having one ridge at each of the proximal end and the distal end.

21. The method of claim 19 wherein the balloon body between the ridges is 6 mm to 30 mm in length.

22. A method of making a zero-fold dilatation balloon, the method comprising:
providing a tubular parison comprising a polymeric material;
providing a mold for forming a balloon with one or more ridges at each of the proximal and distal ends;
expanding the tubular parison to form an expanded parison in the mold;
providing a heat deflector in proximity to the expanded parison to shield a region between the ridges at the proximal end and the distal end of the expanded parison;
subjecting the expanded parison with the shielded region to a shrinkage process to form a zero-fold balloon having a uniform profile along its entire length in a deflated state, and comprising a balloon body having a continuous polymer tube with an external surface, at least one ridge at the proximal end, and at least one ridge at the distal end when in an inflated state; and
applying a hydrophilic coating to the external surface of the continuous polymer tube between the regions at the proximal end and the distal end that form the ridges.

23. The method of claim 22 wherein:
expanding the tubular parison to form an expanded parison comprises axially stretching and radially expanding the tubular parison at a temperature above the Tg of the polymeric material and at an elevated inflation pressure; and
subjecting the expanded parison with the shielded region to a shrinkage process comprises:
heating the expanded parison to a temperature above the temperature at which the balloon was axially stretched and radially expanded, but below the melting temperature of the polymeric material of the tubular parison; and
reducing the inflation pressure to 0 psi;
wherein the shrinkage process is carried out for a time sufficient to form a zero-fold balloon having a uniform profile along its entire length in a deflated state.
